SEQUENCE LISTING (13 sequences)

SEQ ID NO: 1 - DNA, 2500 bp, Sorghum bicolor
attaccaagaatcctcctgacagatccggaaaccaatcgaagattgactccttctagttctaatcctatgtagctatgctaatctagatgtctaattgatgtggtggctctaatcttgatcagaggcttcttctcccttgatggaacaatgaattagggttgagaggccctctcctccaggggccagggggtctggttttatagtccctccaagtgaatatgggcccttggatcaaaccgacatagattgtatggtttagattcatcctttaggtcggtggagttctcccgcaaagcagagtcctgattggacacaggaggtgggcgggcgcccaaggcaggagggcgggcgccctgcctctggccccgtttcgcctccgcttcggtctcgtggcttctggagtcttctagatgtaagataattgcgcagcacgttaatatctttacgtaatcccgacatgtgggcctttcttccatatttcctgataaccccctgcagaaatagacaaacaccaaaacttgtggtattctgtcagataaaaccctaagtctggatcttgatttcatttggatccttttctttatttatttgataattaaatttgatacttaaggaccgtcaacacacaagacacagaccaaatttttatccaggttcggccgccgtgcgggcgtaatacctacgtcctgcgtctgattgtattggattgtgttgagatgagatgtgtatgacctgtcctttaggggacccctgcccctccttatatattgtgaagggacagagttacaagtaaactatcctatttggtacaatatcttgtagccttgcggtgcacgccgaccagcgtcatgcgccgcacgtcttcatcttgtgggccgagccacttctgatggtgcggcccatatcggcccatgagggtataggggtttatacccccacagtgatggcaatcaccaagagtaacaagaaatccccaatcacaaagatttcctagtgccaccaaatccaaatccaaaatgcacatactaggctctcactccctcactcaaaatagcaccaagcatgagagggagaggaaggggaaatggtggatcttcaacctctcatccacacccacaaagggctctcaatctctctaagaaatttcaaccctaacttccttgtgttccaaatggggagagaagtgaggaggaagaaggcagctgcagccccaatcacttagctcgcgctcccaacgatatttatagagtggcaccggatgttccggtgcctagaaccagaacttccggtccaactagttctgaccgttggagcctcggtctaagcagttctgaccgttgtggccaagcagttctaaccgttgtgagagactcggatccatcggaagttccaccggaactttcggttagagaccggaagtgaatcctaacatgcgagggcaagcacaaaggctcctgtgcggacttccgtttatagaccagaacttccagttgtggaccgaaacttctggtcatggaccggaacttccggtcagacaccagaagtgttttctcaaacgtgagggcacctctcacagcccaaccagtttctgtcaggctgactagaacttccggtcagacaccgaaacttccggtcgtgtattcttagcaccatctcaaaacttgttaggatgctaacttctagctccaaactccgaattggatgatcttggacatttcggatagcttactcagagggatatacaatccatatggatactcaatccaagtcataatggatcaaagcagtatttcaatctaaatgtcaccctaatgtccggagaactccgaaaagcctctctctcttctgcaagttgtcactcaattaccaaaactaaataaggacctttcacctagttactctatgattagacaatgtttgtcaaataaaaatgaaagtgctatgatgtcaaaatccaaaaatattttggatctaaacaaggccttagttcactccgaaaccaaaaaagttttcaagattccacgtcttatcgaatcttatggcaaatgcatgaagcattaaatatagataaaaacaaaaactaagtatatattttatccataaattgtaagacgaatatttaagtctagttactctatcattaaatatttttttttaaataaaaacaaaagtgttatagtctcgaaatcaaaaaaaacttttgaatctagacaaggcctaaacaaggccataattgaaaaaaaaaagctggccaatcgccgatgccagtctggccccaacggcactcccacatggcctcacctcacccacccccaccaatcccccgctcagatccgacggccgctaatcgtcgcatccgcaaactaacggtatcgcgccctcgtctctccccataaaaccctcctcacccctgcctcccaacgccgccgtctcctcccctcctcccattccgtcccccctgcccagatctaattctcgacttctccgtccgccgctgcc

SEQ ID NO: 2 - DNA, 2500 bp, Zea mays
gagcgttgacccagccactgagctggtgaagcagtgggtccgcgaggtgggtgatggccctgcccctcgggctggtgaagcagcgagtcggcgaggtgagcgatgacccagcccctgggtcttcaattcaaaattgtgtataaacatggtaagaaaaatgtggtggctaatgctttgtctcgagtgggctttgcaatgaatattacttgtgggtacaagaggtgcttaattcctatgtgacagattctcagtcctaagtgatgctccagaaattggttgttcatagtcgaggagggttatttcttgcacaagggtctgattagaaaaggccagcggatttggaatagggagaattgagctctgtgcacaaggttgatatctgcttttcatgagtcaacattggaaggtcactcaggtatgagtgctacttttcaatgcatcaagaagttcttttgttggaggggtatgaagacagaattggagttgtttgttaggcagtgtggaatatgccggaaagctaagattgagaggcattatcctgctgggctgcttcagccactacctgttccagcaggggcatgggaggacatttctatggatttcattgaaaaactgccccaagttagaaggttttcacacaattattatggtggtggacaaatttcaaagtatacccacttctttccacttaagcatcctttcacaactcaaggtgtggcacaagtgatacttgataatgtggttaagctacatgggctccctaagtcaattgtgtgtgatagagataagatcatcacaagtaacttctagaatcatttgttcaagttgttggagatgaagttggctcttagcaccgtctaccatcctcaaacatatggtcaaagtgaagagtgaatcaaagtctagagatgtatcttagatgtgctgcttctgaaaatccacagaaatggaaaacttggttggctttggcagagttttggtataacacatcatatcatgtgtctttgagatgttctcctttcaaggctttgtatggatatgatgcttctatgatctggcacgatctatgaagggtgatgaagatgtggtttctagagatgtggtgcaacagatagcagtgcatagttctatgctcaaggaacatttgacgaaagtccaacaaaggtttaaacattatgctgatatgagaaggttgcctagggatttcaaattgaagaagaggtgttgctcaagttatagcctacacacaacagtctgtggttagcaaacaatgtccaaagttttcttttcgatattttggaccatacaaggtgttggagaaaattgggaagtggcttacaagttggatctaccagcagactctcaaatacacctagtgtttcatgtgtcacagctaaagccctttgtgcctcgttatacaccggtgttcaagaaccttcctgctatggtggatctagagaatgttgagccaggtaaagtgctagaccgaagattggttaagaaggggaatgcaaccattactcaggttctggttaggtggaccagtccatctccagttcttgcaacatgggaagattaccatgtcttgaaggctcgttttctagctgttcttgcttgaggagaagcaagatttgaagggggatgtaaggcctggtgatattgctaccttttggtgttagggtttgggtagtattttgataatattttgtttgtaatccattagtggaataatgaccggtttctagaagtagaaaggggtattacactatcgtggtagtaggtgagttcatccgtccaatctgtgccactggaagagaggacagggaggagtggaggtgcgacgaccaatggtggtggcggcgacggctggaagaggagagcaaggagaatgagagaggatgaagcatgaatcgggctgtgtgacgggagagcaggtatttttaattggaccactgtttattttgagaaatcggagggtgtttttgcgaaccaagtctcgagatgtgtcagtatatgtataacgtgtttggtttgaggaataagttagtctattatcttctcactcctcactttcttgtttggtttgtaaaatataatggttttatccgtcactatttcctttatttggtaataattagtgataattaaaaaaataagttcatttcactaaatttataaaataaactcataatgcacgaacttatataaaatagattgattccatcattgctcaccaatgtctcagatccgcgcatctcacctgacgtcttcacccaccaacggcactccggtatggcgactcaccaaccccccaccaatcccccgcccagatccgacggccgctaatcgccgcgtccgcaaaccaacggtctggcgccgtcgtctcttcccataaaaccctccccacccctgcctcccaaccgcgccgtcttctccctcactcccgttccatttccccatctcccagatctaattcgcgagttctccctcctctgccgcc

SEQ ID NO: 3 - DNA, 2419 bp, Sorghum bicolor
attaccaagaatcctcctgacagatccggaaaccaatcgaagattgactccttctagttctaatcctatgtagctatgctaatctagatgtctaattgatgtggtggctctaatcttgatcagaggcttcttctcccttgatggaacaatgaattagggttgagaggccctctcctccaggggccagggggtctggttttatagtccctccaagtgaatatgggcccttggatcaaaccgacatagattgtatggtttagattcatcctttaggtcggtggagttctcccgcaaagcagagtcctgattggacacaggaggtgggcgggcgcccaaggcaggagggcgggcgccctgcctctggccccgtttcgcctccgcttcggtctcgtggcttctggagtcttctagatgtaagataattgcgcagcacgttaatatctttacgtaatcccgacatgtgggcctttcttccatatttcctgataaccccctgcagaaatagacaaacaccaaaacttgtggtattctgtcagataaaaccctaagtctggatcttgatttcatttggatccttttctttatttatttgataattaaatttgatacttaaggaccgtcaacacacaagacacagaccaaatttttatccaggttcggccgccgtgcgggcgtaatacctacgtcctgcgtctgattgtattggattgtgttgagatgagatgtgtatgacctgtcctttaggggacccctgcccctccttatatattgtgaagggacagagttacaagtaaactatcctatttggtacaatatcttgtagccttgcggtgcacgccgaccagcgtcatgcgccgcacgtcttcatcttgtgggccgagccacttctgatggtgcggcccatatcggcccatgagggtataggggtttatacccccacagtgatggcaatcaccaagagtaacaagaaatccccaatcacaaagatttcctagtgccaccaaatccaaatccaaaatgcacatactaggctctcactccctcactcaaaatagcaccaagcatgagagggagaggaaggggaaatggtggatcttcaacctctcatccacacccacaaagggctctcaatctctctaagaaatttcaaccctaacttccttgtgttccaaatggggagagaagtgaggaggaagaaggcagctgcagccccaatcacttagctcgcgctcccaacgatatttatagagtggcaccggatgttccggtgcctagaaccagaacttccggtccaactagttctgaccgttggagcctcggtctaagcagttctgaccgttgtggccaagcagttctaaccgttgtgagagactcggatccatcggaagttccaccggaactttcggttagagaccggaagtgaatcctaacatgcgagggcaagcacaaaggctcctgtgcggacttccgtttatagaccagaacttccagttgtggaccgaaacttctggtcatggaccggaacttccggtcagacaccagaagtgttttctcaaacgtgagggcacctctcacagcccaaccagtttctgtcaggctgactagaacttccggtcagacaccgaaacttccggtcgtgtattcttagcaccatctcaaaacttgttaggatgctaacttctagctccaaactccgaattggatgatcttggacatttcggatagcttactcagagggatatacaatccatatggatactcaatccaagtcataatggatcaaagcagtatttcaatctaaatgtcaccctaatgtccggagaactccgaaaagcctctctctcttctgcaagttgtcactcaattaccaaaactaaataaggacctttcacctagttactctatgattagacaatgtttgtcaaataaaaatgaaagtgctatgatgtcaaaatccaaaaatattttggatctaaacaaggccttagttcactccgaaaccaaaaaagttttcaagattccacgtcttatcgaatcttatggcaaatgcatgaagcattaaatatagataaaaacaaaaactaagtatatattttatccataaattgtaagacgaatatttaagtctagttactctatcattaaatatttttttttaaataaaaacaaaagtgttatagtctcgaaatcaaaaaaaacttttgaatctagacaaggcctaaacaaggccataattgaaaaaaaaaagctggccaatcgccgatgccagtctggccccaacggcactcccacatggcctcacctcacccacccccaccaatcccccgctcagatccgacggccgctaatcgtcgcatccgcaaactaacggtatcgcgccctcgtctctccccataaaaccctcctcacccctgcctcc

SEQ ID NO: 4 - DNA, 2417 bp, Zea mays
gagcgttgacccagccactgagctggtgaagcagtgggtccgcgaggtgggtgatggccctgcccctcgggctggtgaagcagcgagtcggcgaggtgagcgatgacccagcccctgggtcttcaattcaaaattgtgtataaacatggtaagaaaaatgtggtggctaatgctttgtctcgagtgggctttgcaatgaatattacttgtgggtacaagaggtgcttaattcctatgtgacagattctcagtcctaagtgatgctccagaaattggttgttcatagtcgaggagggttatttcttgcacaagggtctgattagaaaaggccagcggatttggaatagggagaattgagctctgtgcacaaggttgatatctgcttttcatgagtcaacattggaaggtcactcaggtatgagtgctacttttcaatgcatcaagaagttcttttgttggaggggtatgaagacagaattggagttgtttgttaggcagtgtggaatatgccggaaagctaagattgagaggcattatcctgctgggctgcttcagccactacctgttccagcaggggcatgggaggacatttctatggatttcattgaaaaactgccccaagttagaaggttttcacacaattattatggtggtggacaaatttcaaagtatacccacttctttccacttaagcatcctttcacaactcaaggtgtggcacaagtgatacttgataatgtggttaagctacatgggctccctaagtcaattgtgtgtgatagagataagatcatcacaagtaacttctagaatcatttgttcaagttgttggagatgaagttggctcttagcaccgtctaccatcctcaaacatatggtcaaagtgaagagtgaatcaaagtctagagatgtatcttagatgtgctgcttctgaaaatccacagaaatggaaaacttggttggctttggcagagttttggtataacacatcatatcatgtgtctttgagatgttctcctttcaaggctttgtatggatatgatgcttctatgatctggcacgatctatgaagggtgatgaagatgtggtttctagagatgtggtgcaacagatagcagtgcatagttctatgctcaaggaacatttgacgaaagtccaacaaaggtttaaacattatgctgatatgagaaggttgcctagggatttcaaattgaagaagaggtgttgctcaagttatagcctacacacaacagtctgtggttagcaaacaatgtccaaagttttcttttcgatattttggaccatacaaggtgttggagaaaattgggaagtggcttacaagttggatctaccagcagactctcaaatacacctagtgtttcatgtgtcacagctaaagccctttgtgcctcgttatacaccggtgttcaagaaccttcctgctatggtggatctagagaatgttgagccaggtaaagtgctagaccgaagattggttaagaaggggaatgcaaccattactcaggttctggttaggtggaccagtccatctccagttcttgcaacatgggaagattaccatgtcttgaaggctcgttttctagctgttcttgcttgaggagaagcaagatttgaagggggatgtaaggcctggtgatattgctaccttttggtgttagggtttgggtagtattttgataatattttgtttgtaatccattagtggaataatgaccggtttctagaagtagaaaggggtattacactatcgtggtagtaggtgagttcatccgtccaatctgtgccactggaagagaggacagggaggagtggaggtgcgacgaccaatggtggtggcggcgacggctggaagaggagagcaaggagaatgagagaggatgaagcatgaatcgggctgtgtgacgggagagcaggtatttttaattggaccactgtttattttgagaaatcggagggtgtttttgcgaaccaagtctcgagatgtgtcagtatatgtataacgtgtttggtttgaggaataagttagtctattatcttctcactcctcactttcttgtttggtttgtaaaatataatggttttatccgtcactatttcctttatttggtaataattagtgataattaaaaaaataagttcatttcactaaatttataaaataaactcataatgcacgaacttatataaaatagattgattccatcattgctcaccaatgtctcagatccgcgcatctcacctgacgtcttcacccaccaacggcactccggtatggcgactcaccaaccccccaccaatcccccgcccagatccgacggccgctaatcgccgcgtccgcaaaccaacggtctggcgccgtcgtctcttcccataaaaccctccccacccctgcctcc

SEQ ID NO: 5 - DNA, 96 bp, Artificial Sequence
  attL4 site in vector PHP56634
aaataatgattttattttgactgatagtgacctgttcgttgcaacaaattgataagcaatgcttttttataatgccaactttgtatagaaaagttg

SEQ ID NO: 6 - DNA, 2001 bp, Artificial Sequence
  Sequence encoding GUS with intron added artificially
atggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 7 - DNA, 310 bp, Solanum tuberosum
cctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttg

SEQ ID NO: 8 - DNA, 1043 bp, Sorghum bicolor
gtttttctcagacagttttctaaaaaaagggcgtttctggggaagttcgagatggttcgtaaggtgttactggctcctgtgaaccaatacatgatactgccatgataagggttataattagtcaagcagagtaagaagaaacaacagtagcagtgactccgattcctgaagatgagtcatatttgtcttgtgctcctgctgtatgaaatggatcgcatgtgtatattcgtcgccgcgccgcactggtgtaacctgttgcctcagagtttgcttttagctggttctgttttaaaaataagtactgttttttggttggctgcaagccattctgaacttcagtttaccaattgtttttatgttgtggttgaatattttaattttttatttaatgtttggttctttttttatatatatttgcaaaaatgatacaagtggtcaagttttcatatagtatgggctctatttcctagagctctacctctaggaacgaattttgtggaggttttcttttggctagttaggcaaagtccccatatcttgcaggctaaatcaagaagaagctctgtcaaacagttttttttactgaaaagtgattaaagagtagtttctcctagatcacttcagagtttatcctagagaatcatgggaatcaaattcagttagaggatcatttcttacaaagaatcaactttcgtagagaatctaaagcagaaagagctttgacaaacttacccttagagcaattccaacattctcgcgtgagtttcttcgcgccgttgttttgcggtgacttcatctggacgtcccgcgacatagagacgcttgtattgatcatgagagcttgtgtggtcatacacaatataattgttaaagatgaaagagatgtggaccttaatgagcgattcgactttgatggtgaaaatgtgcaaccttctcatggtatttctactcgcacactagctgaatttattgaagctcataaaaagatccgagacaaagaaatacattttcaattgaaagaagacctaatcaagcacttatgg

SEQ ID NO: 9 - DNA, 1991 bp, Zea mays
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 10 - DNA, 1827 bp, Zea mays
cctggtaattattggctgtaggattctaaacagagcctaaatagctggaatagctctagccctcaatccaaactaatgatatctatacttatgcaactctaaatttttattctaaaagtaatatttcatttttgtcaacgagattctctactctattccacaatcttttgaagctatatttaccttaaatctgtactctataccaataatcatatattctattatttatttttatctctctcctaaggagcatccctctatgtctgcatggcccccgcctcgggtcccaatctcttgctctgctagtagcacagaagaaaacactagaaatgacttgcttgacttagagtatcagataaacatcatgtttacttaactttaatttgtatcggtttctactatttttataatatttttgtctctatagatactacgtgcaacagtataatcaacctagtttaatccagagcgaaggattttttactaagtacgtgactccatatgcacagcgttccttttatggttcctcactgggcacagcataaacgaaccctgtccaatgttttcagcgcgaacaaacagaaattccatcagcgaacaaacaacatacatgcgagatgaaaataaataataaaaaaagctccgtctcgataggccggcacgaatcgagagcctccatagccagttttttccatcggaacggcggttcgcgcacctaattatatgcaccacacgcctataaagccaaccaacccgtcggaggggcgcaagccagacagaagacagcccgtcagcccctctcgtttttcatccgccttcgcctccaaccgcgtgcgctccacgcctcctccaggaaagcgagaggtgagcgcagtcccctttcccctccttccaattcaattcgtcttctcgttcgcagccctaggatttgggggtctggaggggtttgatcgtttctcgccgtgaatctgctttggtgtaaaccaacggatctcggatcgtagtcttcagaagatcccggattttgcggtttggcccctcctggattcaattcgtcgtatcgttcgcagccctaggatttggggatctggaggggtttgatcgtttctcgccgcgaatctgctctggtgtaaaccaacggatctcgggtcgtagtcttcagaaggtcccggattttgcggtttggcccctcctggattcaattcgtcgtatcgttcgcagccctaggatttggggatctggaggggtttgatcctttctcgccgcgaatctgctctggtataaccaacggatctcgggtcgtagtcttcagaaggtcccggattttgcggtttggtggttcttgctctatgaatcagagggatggttcttcccggatttatgccttgcggccactctgtcgaatcatggggtttcgacccgattcgtaggcgtgctccctgttttggatgggaagtaggcgtgtttgtagtattcgtgcttcgattcgtcaacggagattagaagacctgggatgggatttgaggaaatctaggtatctgtctagcacgtttctagatctattcttcagctgttatatgagagtaattttggaaccctggtggggtatgtttgaccgagtattctgtagattattgtccgtgacttgctggctgttaccgtccttatctctgcatcatctatctgtgctagtttctgcgtgcttctcaaatatttccggcctgtgtagcatgtgactgataatatgattttggcagcttctgcataagaacaacaaatcaaaagcttgatcagctcggtgcctacaaaacctcaacaaccaagttt

SEQ ID NO: 11 - DNA, 535 bp, Zea mays
gtccgccttgtttctcctctgtctcttgatctgactaatcttggtttatgattcgttgagtaattttggggaaagcttcgtccacagttttttttcgatgaacagtgccgcagtggcgctgatcttgtatgctatcctgcaatcgtggtgaacttatttcttttatatcctttactcccatgaaaaggctagtaatctttctcgatgtaacatcgtccagcactgctattaccgtgtggtccatccgacagtctggctgaacacatcatacgatctatggagcaaaaatctatcttccctgttctttaatgaaggacgtcattttcattagtatgatctaggaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctgaacccgtgcag

SEQ ID NO: 12 - DNA, 856 bp, Zea mays
gtaaggttcccttccctcctcccctcacacccctgttcgtgttccttcggatcggatctcagtggtgatgttagacgtccgcggctgcctacgtagtggcattgccgcccgaaaggtttgtttaggtggggtagatccgaaacaggccggatctggaccatgtccgcggcggggcggcgggacttgatcgcgtagctgtcgtgtgcatttctccctaccagtggcggaatcggcgatgtggacctaagggctaaggcttatctgctgccttgaccatttcgtcgctgacaaaaacaaagtgacaatcatgccgttctctgtttgtttatctggatcgttattacgctgtgaatcctgcgatatgtggctaagtgatttttcttctttttctgggggcagtttagcctttgacccagtcctaggtgtggtcactaggactgtgtagcatgatgagtgaggttgcagcaggctgattgctagtggacgtttttttccccaatttgttaggttttcacgctccaggttgtgcaagtaattttgctagtgattgtgtgatccatcttcaacgttgaaccttgtttttccccctaaaacccccaacaggaaatcttgccccgacttctattgcaaaaattgtaacgcttagcaccctgattgactcaattcctgtcactaggcatgctcggtcaaaagcagatgatttaccacttagaaactgccctgcccctgctttccacatagcatttcgaactttttgactactattgacacccccctaacttgccgaactatttctctcttcagctactatttacctagttataattacataaatgtttgtgtgtatcttgtgcag

SEQ ID NO: 13 - DNA, 884 bp, Zea mays
gtgaggcatccgatcgatttttctttctttctttactacactccttcgcgatatggggacggcactcggtagtggcgtgaggtgcggtaaatcgcgttagtttagttgtagggtttgatcgcttcgggggggaccgggggttgggcttcccgtgttgaaccgtcaatcggacgtagtagtagtgcggattcggggtttgatcgacggaaagaggggttgtccgcactcttggtgtggttatagggttttgcgatttgtttgtctgtgtaggcccgtttcgtctcggggagtagattttcattgctactaccaatccctatgtgctttggtgaacacgtattttggtctgtatatggtttaaacgtgaagactatggtagtgtgagaccatgatttggatccttttctgtggcattatagttaaaatcgtgaggatctatcttttagcgcttagggtattgttatagacgagatcccctctttgggctctaaaaatagcaagaaaaggacatcttttgggcaagttaacgtcctgtattattctgaacgagatctgtttactttcttataagtttgatgttttggtctggaatatggttgcgttcatcgtccaattagtgtgtttgcagtatgtgttggtgtagttcctctgtgggcattttgtggccccagaaatgatagattttaagaaaggtttaggcagaaggggatcttaagtgttgtccagtacaaagtaacaatttgtagcacttgtttcttttcttttgtttgactatatgaaatttcggccatgtaattgtttcaaaataataagatcgaatagtgttgcacactacttcccagtcctatgtatacttatcagatttttcctctttgatatttcag

(19) United States
(12) Patent Application Publication
(10) Pub. No.: US 2018/0265881 A1
(43) Pub. Date: Sep. 20, 2018

(54) MAIZE AND SORGHUM S-ADENOSYL-HOMOCYSTEINE HYDROLASE PROMOTERS

(71) Applicant: PIONEER HI-BRED INTERNATIONAL, INC., Johnston, IA (US)

(72) Inventors: Shane E. Abbitt, ANKENY, IA (US); BO SHEN, JOHNSTON, IA (US)

(21) Appl. No.: 15/540,091

(22) PCT Filed: Dec. 11, 2015
(86) PCT No.: PCT/US15/65188

§ 371 Date: Jun. 27, 2017

Related U.S. Application Data
(60) Provisional application No. 62/097,514, filed on Dec. 29, 2014.

(51) Int. Cl. C12N 15/82; C07K 14/415

(52) U.S. Cl. CPC C12N 15/8217; C12N 15/8226; C07K 14/415; C12N 15/8231; C12N 15/8225; C12N 15/8227

(57) ABSTRACT

The disclosure describes promoter sequences, specifically S-Adenosyl-Homocysteine Hydrolase promoter sequences from maize and sorghum, which are useful for expression of transgenes in plants. The disclosure further describes isolated polynucleotides, recombinant DNA constructs, transformed host cells, transgenic plants and transgenic seeds, and corresponding methods of use of the promoter sequences.

### CROSS REFERENCE TO RELATED APPLICATIONS

This application claims the benefit of U.S. Provisional Application No. 62/097,514, filed Dec. 29, 2014, the entire content of which is herein incorporated by reference.

### REFERENCE TO SEQUENCE LISTING SUBMITTED ELECTRONICALLY

The official copy of the sequence listing is submitted electronically via EFS-Web as an ASCII formatted sequence listing with a file named “20151203_BB2401PCT_SequenceListing.txt” created on Dec. 3, 2015, and having a size of 26 kilobytes and is filed concurrently with the specification. The sequence listing contained in this ASCII formatted document is part of the specification and is herein incorporated by reference in its entirety.

### FIELD

The present present disclosure relates to the field of plant molecular biology and plant genetic engineering. More specifically, it relates to compositions and the use of regulatory sequences such as promoter sequences to regulate gene expression in plants.

### BACKGROUND

Recent advances in plant genetic engineering have created opportunities to engineer plants to have improved characteristics or traits. These transgenic plants characteristically have recombinant DNA constructs in their genome that have a polynucleotide of interest operably linked to at least one regulatory region, e.g., a promoter that allows expression of the transgene. The expression level of the polynucleotide of interest can also be modulated by other regulatory elements such as introns and enhancers.

Promoters can be strong or weak promoters, or can be constitutive, tissue-specific or might be regulated in a spatiotemporal or inducible manner. Thus, promoters allow transgene expression to be regulated, allowing for more precise control over the manner in which the transgene, and hence the phenotype conferred by it, is expressed. Plant genetic engineering has advanced to introducing multiple traits into commercially important plants, also known as gene stacking. This is accomplished by multigene transformation, where multiple genes are transferred to create a transgenic plant that might express a complex phenotype, or multiple phenotypes. But it is important to modulate or control the expression of each transgene optimally, and the regulatory elements need to be diverse, to avoid introducing into the same transgenic plant repetitive sequences, which have been correlated with undesirable negative effects on transgene expression and stability (Peremarti et al (2010) Plant Mol Biol 73:363-378; Mette et al (1999) EMBO J 18:241-248; Mette et al (2000) EMBO J 19:5194-5201; Mourrain et al (2007) Planta 225:365-379, U.S. Pat. No. 7,632.982, U.S. Pat. No. 7,491,813, U.S. Pat. No. 7,674,950, PCT Application No. PCT/US2009/046968). Therefore it is important to discover and characterize novel regulatory elements that can be used to express heterologous nucleic acids in important crop species. Diverse promoters with desired expression profiles can be used to control the expression of each transgene optimally.

### SUMMARY

The present disclosure includes novel regulatory sequences from sorghum and maize, i.e., promoter and 5′ untranslated leader regions, which can be used for regulating gene expression of heterologous polynucleotides in transgenic plants.

The present disclosure provides a recombinant DNA construct comprising a promoter functional in a plant cell operably linked to a heterologous polynucleotide, wherein the promoter comprises a nucleotide sequence selected from the group consisting of: (a) a nucleotide sequence with at least 90% sequence identity to SEQ ID NO:1, 2, 3 or 4, using a Clustal V method of alignment with pairwise alignment default parameters of KTUPLE=2, GAP PENALTY=5, WINDOW=4 and DIAGONALS SAVED=4; (b) a nucleotide sequence comprising a functional fragment of SEQ ID NO:1, 2, 3 or 4, wherein the functional fragment comprises at least 100 contiguous nucleotides of SEQ ID NO:1, 2, 3 or 4; and (c) SEQ ID NO:1, 2, 3 or 4; wherein the promoter drives expression of the heterologous polynucleotide in a plant cell.

The promoter of the recombinant DNA construct may drive expression in at least one tissue selected from the group consisting of: anther, leaf, root and stalk. The promoter of the recombinant DNA construct may drive expression in anther, leaf, root and stalk.

The promoter of the recombinant DNA construct may drive expression of the heterologous polynucleotide primarily in plant vascular cells.

The promoter and the heterologous polynucleotide of the recombinant DNA construct may each be operably linked to an intron.

The promoter of the recombinant DNA construct may not be operably linked to a heterologous enhancer.

The present disclosure also includes a plant or seed comprising the recombinant DNA construct. The plant or seed may be a monocot plant or monocot seed. The monocot plant or monocot seed may be a maize plant or maize seed. The present disclosure provides a method of making a transgenic plant, the method comprising the steps of: (a) introducing into a regenerable plant cell the recombinant DNA construct of the present disclosure; and (b) regenerating a transgenic plant from the regenerable plant cell after step (a), wherein the transgenic plant comprises the recombinant DNA construct. The method may further comprise growing the transgenic plant of step (b) under conditions in which the heterologous polynucleotide is expressed. The method may further comprise growing a progeny plant derived from the transgenic plant of step (b), wherein said progeny plant comprises the recombinant DNA construct and wherein the progeny plant is grown under conditions in which the heterologous polynucleotide is expressed. In any of the methods of the present disclosure, the plant may be a monocot plant. Additionally, the monocot plant may be a maize plant.

The present disclosure also provides a plant comprising in its genome an endogenous polynucleotide operably linked to at least one heterologous promoter functional in a plant cell, wherein the promoter comprises a nucleotide sequence selected from the group consisting of: (a) a nucleotide sequence with at least 90% sequence identity to SEQ ID NO:1, 2, 3 or 4, using a Clustal V method of alignment with pairwise alignment default parameters of KTUPLE=2, GAP PENALTY=5, WINDOW=4 and DIAGONALS SAVED=4; (b) a nucleotide sequence comprising a functional fragment of SEQ ID NO:1, 2, 3 or 4, wherein the functional fragment comprises at least 100 contiguous nucleotides of SEQ ID NO:1, 2, 3 or 4; and (c) SEQ ID NO:1, 2, 3 or 4; wherein said plant exhibits at least one trait selected from the group consisting of: increased drought tolerance, increased yield, increased biomass, and altered root architecture, when compared to a control plant not comprising the heterologous regulatory element.

The present disclosure also provides for the use of any of the recombinant DNA constructs of the present disclosure to produce a plant that exhibits at least one trait selected from the group consisting of: increased drought tolerance, increased yield, increased biomass, and altered root architecture, when compared to a control plant not comprising the heterologous regulatory element.

### BRIEF DESCRIPTION OF THE SEQUENCE LISTING

The present disclosure can be more fully understood from the following detailed description and the accompanying Sequence Listing which form a part of this application.

SEQ ID NO:1 is the nucleotide sequence of Sorghum bicolor S-Adenosyl-Homocysteine Hydrolase (SB-S2A) promoter, upstream of the translation start codon, and including the 5′UTR.

SEQ ID NO:2 is the nucleotide sequence of Zea mays S-Adenosyl-Homocysteine Hydrolase (ZM-S2A) promoter, upstream of the translation start codon, and including the 5′UTR.

SEQ ID NO:3 is the nucleotide sequence of Sorghum bicolor S-Adenosyl-Homocysteine Hydrolase (SB-S2A) promoter, upstream of the predicted transcription start site.

SEQ ID NO:4 is the nucleotide sequence of Zea mays S2A S-Adenosyl-Homocysteine Hydrolase (ZM-S2A) promoter, upstream of the predicted transcription start site.

SEQ ID NO:5 is the nucleotide sequence of the attL4 site present in entry clone PHP56634.

SEQ ID NO:6 is the nucleotide sequence of the β-glucuronidase (GUS) coding region present in entry clone PHP56634.

SEQ ID NO:7 is the nucleotide sequence of the potato pinII terminator present in entry clone PHP56634.

SEQ ID NO:8 is the nucleotide sequence of the Sorghum actin terminator present in entry clone PHP56634.

SEQ ID NO:9 is the nucleotide sequence of the maize Ubi1 promoter and intron present in expression vector PHP49982.

SEQ ID NO:10 is the nucleotide sequence of the maize GOS2 promoter and intron present in expression vector PHP55515.

SEQ ID NO:11 is the nucleotide sequence of a maize intron, ZM-Adh1 intron1, present in Table 4.

SEQ ID NO:12 is the nucleotide sequence of a maize intron, ZM-HPLV9 intron1, present in Table 4.

SEQ ID NO:13 is the nucleotide sequence of a maize intron, ZM-TA4 intron1, present in Table 4.

The sequence descriptions and Sequence Listing attached hereto comply with the rules governing nucleotide and/or amino acid sequence disclosures in patent applications as set forth in 37 C.F.R. § 1.821-1.825.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. § 1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

The present disclosure describes novel regulatory sequences from sorghum and maize that can be used for regulating gene expression of heterologous polynucleotides in transgenic plants. It discloses Sorghum and maize promoter and 5′ untranslated leader sequences that can be used to regulate plant gene expression of heterologous polynucleotides.

The terms “monocot” and “monocotyledonous plant” are used interchangeably herein. A monocot of the current disclosure includes the Gramineae.

The terms “dicot” and “dicotyledonous plant” are used interchangeably herein. A dicot of the current disclosure includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Agronomic characteristic” is a measurable parameter including but not limited to, abiotic stress tolerance, greenness, stay-green, yield, growth rate, biomass, fresh weight at maturation, dry weight at maturation, fruit yield, seed yield, seed size, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen stress tolerance, nitrogen uptake, standability, root lodging, root architecture, root mass, average root length, leaf size, harvest index, stalk lodging, plant height, ear height, ear length, ear size, endosperm size, embryo size, salt tolerance, early seedling vigor and seedling emergence under low temperature stress. The terms “agronomic characteristic” and “agronomic trait” are used interchangeably herein.

Yield can be measured in many ways, including, for example, test weight, seed weight, seed number per plant, seed number per unit area (i.e. seeds, or weight of seeds, per acre), bushels per acre, tonnes per hectare, tonnes per acre, tons per acre and kilograms per hectare.

Abiotic stress may be at least one condition selected from the group consisting of: drought, water deprivation, flood, high light intensity, high temperature, low temperature, salinity, etiolation, defoliation, heavy metal toxicity, anaerobiosis, nutrient deficiency, nutrient excess, UV irradiation, atmospheric pollution (e.g., ozone) and exposure to chemicals (e.g., paraquat) that induce production of reactive oxygen species (ROS). Nutrients include, but are not limited to, the following: nitrogen (N), phosphorus (P), potassium (K), calcium (Ca), magnesium (Mg) and sulfur (S).

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions.

A plant with “increased stress tolerance” can exhibit increased tolerance to one or more different stress conditions.

“Stress tolerance activity” of a polypeptide indicates that over-expression of the polypeptide in a transgenic plant confers increased stress tolerance to the transgenic plant relative to a reference or control plant.

Increased biomass can be measured, for example, as an increase in plant height, plant total leaf area, plant fresh weight, plant dry weight or plant seed yield, as compared with control plants.

The ability to increase the biomass or size of a plant would have several important commercial applications. Crop species may be generated that produce larger cultivars, generating higher yield in, for example, plants in which the vegetative portion of the plant is useful as food, biofuel or both.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell.

“cDNA” refers to a DNA that is complementary to and synthesized from a mRNA template using the enzyme reverse transcriptase. The cDNA can be single-stranded or converted into the double-stranded form using the Klenow fragment of DNA polymerase I.

“Coding region” refers to the portion of a messenger RNA (or the corresponding portion of another nucleic acid molecule such as a DNA molecule) which encodes a protein or polypeptide. “Non-coding region” refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Mature” protein refers to a post-translationally processed polypeptide; i.e., one from which any pre- or pro-peptides present in the primary translation product have been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein.

The terms “entry clone” and “entry vector” are used interchangeably herein.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation. “Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

As will be evident to one of skill in the art, any heterologous polynucleotide of interest can be operably linked to the regulatory sequences described in the current disclosure. Examples of polynucleotides of interest that can be operably linked to the regulatory elements described in this disclosure include, but are not limited to, polynucleotides comprising other regulatory elements such as introns, enhancers, polyadenylation signals, translation leader sequences, protein coding regions such as disease and insect resistance genes, genes conferring nutritional value, genes conferring yield and heterosis increase, genes conferring resistance to biotic or abiotic stress, genes that can confer alterations in one or more than one agronomic characteristics, genes that confer male and/or female sterility, antifungal, antibacterial or antiviral genes, and the like. Likewise, the promoter and intron sequences described in the current disclosure can be used to modulate the expression of any nucleic acid to control gene expression. Examples of nucleic acids that could be used to control gene expression include, but are not limited to, antisense oligonucleotides, suppression DNA constructs, or nucleic acids encoding transcription factors.

The promoter described in the current disclosure can be operably linked to other regulatory sequences. Examples of such regulatory sequences include, but are not limited to, introns, terminators, enhancers, polyadenylation signal sequences, untranslated leader sequences. The promoter sequence described in the present disclosure can be operably linked to the intronic sequences described herein, but can also be operably linked to other intronic sequences. Other introns are known in art that can enhance gene expression, examples of such introns include, but are not limited to, first intron from Adh1 gene, first intron from Shrunken-1 gene, Callis et al., Genes Dev. 1987 1:1183-1200, Mascarenkas et al., Plant Mol. Biol., 1990, 15: 913-920).

Regulatory Sequences:

A recombinant DNA construct (including a suppression DNA construct) of the present disclosure may comprise at least one regulatory sequence. The regulatory sequences disclosed herein can be operably linked to any other regulatory sequence.

“Regulatory sequences” or “regulatory elements” are used interchangeably and refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Developmentally regulated promoter” refers to a promoter whose activity is determined by developmental events.

Promoters that cause a gene to be expressed in most cell types at most times are commonly referred to as “constitutive promoters”.

Inducible promoters selectively express an operably linked DNA sequence in response to the presence of an endogenous or exogenous stimulus, for example by chemical compounds (chemical inducers) or in response to environmental, hormonal, chemical, and/or developmental signals. Examples of inducible or regulated promoters include, but are not limited to, promoters regulated by light, heat, stress, flooding or drought, pathogens, phytohormones, wounding, or chemicals such as ethanol, jasmonate, salicylic acid, or safeners.

“Tissue-specific promoter” and “tissue-preferred promoter” are used interchangeably, and to refer herein to a promoter that causes an operably linked gene or polynucleotide to be expressed predominantly but not necessarily exclusively in one tissue or organ. A tissue-specific promoter may cause an operably linked gene or polynucleotide to be expressed predominantly in one specific cell.

A vascular promoter is a tissue-specific promoter that leads to an operably linked gene or polynucleotide to be expressed primarily in the vascular tissues of a plant.

The vascular tissue of a plant includes phloem, xylem, sclerenchyma and parenchyma cells, is specialized for mechanical support or for conducting nutrients and water (Abrahams et al Plant Molecular Biology 27: 513-528, 1995).

The terms “S-Adenosyl-Homocysteine Hydrolase” and S2A are used interchangeably herein, and refer to the enzyme S-adenosyl-homocysteine hydrolase, responsible for the hydrolysis of S-adenosyl-L-homocysteine (SAH) to adenosine and homocysteine.

The terms “Sorghum S-adenosyl-homocysteine hydrolase”, “SbS2A” and “SB-S2A” are used interchangeably herein and refer to the S-adenosyl-homocysteine hydrolase enzyme from Sorghum bicolor. The promoter sequence for this gene is given in SEQ ID NOS:1 and 3 in this disclosure.

The terms “Maize S-adenosyl-homocysteine hydrolase”, “ZmS2A” and “ZM-S2A” are used interchangeably herein and refer to the S-adenosyl-homocysteine hydrolase enzyme from Zea mays. The promoter sequence for this gene is given in SEQ ID NOS:2 and 4 in this disclosure.

This pathway for the metabolism of SAH is the only pathway in most species. S2A has been found in all cells tested with the exception of Escherichia coli and other related bacteria (U.S. Pat. No. 6,037,524).

The S2A enzyme (S-adenosyl-homocysteine hydrolase) is responsible for the hydrolysis of S-adenosyl-L-homocysteine to adenosine and homocysteine (Abrahams et al, 1995 Plant Molecular Biology 27: 513-528, 1995). Abrahams et al. (1995) have shown that the Medicago sativa S-adenosyl-homocysteine hydrolase enzyme (MS-S2A) is primarily expressed in mature stem sections, primarily in sclerenchyma cells found around individual vascular bundles and in developing xylem cells in vascular bundles.

The promoter from an Arabidopsis S-adenosyl-L-homocysteine hydrolase gene was described in U.S. Pat. No. 6,037,524, and described as a promoter useful for constitutive expression or to target increased levels of gene expression at sites of wounding or pathogen invasion.

Sujatha et al. have reported (J. Plant Biochemistry & Biotechnology Vol. 18(1), 13-20, January 2009) that a 1585 bp fragment upstream to ATG of one of the S-adenosyl homocysteine hydrolase genes in Arabidopsis ((At4g13940) when fused with a promoter-less β-Glucuronidase (GUS) gene and mobilized into Arabidopsis drove constitutive expression of GUS in T2 progeny of transgenic Arabidopsis. The authors also reported that a 376 bp promoter fragment of this gene, was found to be capable of driving GUS expression in developing seeds and in some anthers/microspores.

A minimal or basal promoter is a polynucleotide molecule that is capable of recruiting and binding the basal transcription machinery. One example of basal transcription machinery in eukaryotic cells is the RNA polymerase II complex and its accessory proteins.

Plant RNA polymerase II promoters, like those of other higher eukaryotes, are comprised of several distinct “cis-acting transcriptional regulatory elements,” or simply “cis-elements,” each of which appears to confer a different aspect of the overall control of gene expression. Examples of such cis-acting elements include, but are not limited to, such as TATA box and CCAAT or AGGA box. The promoter can roughly be divided in two parts: a proximal part, referred to as the core, and a distal part. The proximal part is believed to be responsible for correctly assembling the RNA polymerase II complex at the right position and for directing a basal level of transcription, and is also referred to as “minimal promoter” or “basal promoter”. The distal part of the promoter is believed to contain those elements that regulate the spatio-temporal expression. In addition to the proximal and distal parts, other regulatory regions have also been described, that contain enhancer and/or repressors elements The latter elements can be found from a few kilobase pairs upstream from the transcription start site, in the introns, or even at the 3′ side of the genes they regulate (Rombauts, S. et al. (2003) Plant Physiology 132:1162-1176, Nikolov and Burley, (1997) Proc Natl Acad Sci USA 94: 15-22), Tjian and Maniatis (1994) Cell 77: 5-8; Fessele et al., 2002 Trends Genet 18: 60-63, Messing et al., (1983) Genetic Engineering of Plants: an Agricultural Perspective, Plenum Press, NY, pp 211-227).

When operably linked to a heterologous polynucleotide sequence, a promoter controls the transcription of the linked polynucleotide sequence.

In the present disclosure, the “cis-acting transcriptional regulatory elements” from the promoter sequence disclosed herein may be operably linked to “cis-acting transcriptional regulatory elements” from any heterologous promoter. Such a chimeric promoter molecule can be engineered to have desired regulatory properties. In the present disclosure, a fragment of the disclosed promoter sequence that can act either as a cis-regulatory sequence or a distal-regulatory sequence or as an enhancer sequence or a repressor sequence, may be combined with either a cis-regulatory or a distal regulatory or an enhancer sequence or a repressor sequence or any combination of any of these from a heterologous promoter sequence.

In the present disclosure, a cis-element of the disclosed promoter may confer a particular specificity such as conferring enhanced expression of operably linked polynucleotide molecules in certain tissues and therefore is also capable of regulating transcription of operably linked polynucleotide molecules. Consequently, fragments, portions, or regions of the promoter comprising the polynucleotide sequence shown in SEQ ID NO:1, 2, 3 or 4 can be used as regulatory polynucleotide molecules.

Promoter fragments that comprise regulatory elements can be added, for example, fused to the 5′ end of, or inserted within, another promoter having its own partial or complete regulatory sequences (Fluhr et al., Science 232:1106-1112, 1986; Ellis et al., EMBO J. 6:11-16, 1987; Strittmatter and Chua, Proc. Nat. Acad. Sci. USA 84:8986-8990, 1987; Poulsen and Chua, Mol. Gen. Genet. 214:16-23, 1988; Comai et al., Plant Mol. Biol. 15:373-381, 1991; 1987; Aryan et al., Mol. Gen. Genet. 225:65-71, 1991).

Cis elements can be identified by a number of techniques, including deletion analysis, i.e., deleting one or more nucleotides from the 5′ end or internal to a promoter; DNA binding protein analysis using DNase I footprinting; methylation interference; electrophoresis mobility-shift assays, in vivo genomic footprinting by ligation-mediated PCR; and other conventional assays; or by sequence similarity with known cis element motifs by conventional sequence comparison methods. The fine structure of a cis element can be further studied by mutagenesis (or substitution) of one or more nucleotides or by other conventional methods (see for example, Methods in Plant Biochemistry and Molecular Biology, Dashek, ed., CRC Press, 1997, pp. 397-422; and Methods in Plant Molecular Biology, Maliga et al., eds., Cold Spring Harbor Press, 1995, pp. 233-300).

Cis elements can be obtained by chemical synthesis or by cloning from promoters that include such elements, and they can be synthesized with additional flanking sequences that contain useful restriction enzyme sites to facilitate subsequent manipulation. Promoter fragments may also comprise other regulatory elements such as enhancer domains, which may further be useful for constructing chimeric molecules.

Methods for construction of chimeric and variant promoters of the present disclosure include, but are not limited to, combining control elements of different promoters or duplicating portions or regions of a promoter (see for example, U.S. Pat. No. 4,990,607 U.S. Pat. No. 4,990,607; U.S. Pat. No. 5,110,732 U.S. Pat. No. 5,110,732; and U.S. Pat. No. 5,097,025 U.S. Pat. No. 5,097,025). Those of skill in the art are familiar with the standard resource materials that describe specific conditions and procedures for the construction, manipulation, and isolation of macromolecules (e.g., polynucleotide molecules and plasmids), as well as the generation of recombinant organisms and the screening and isolation of polynucleotide molecules.

In the present disclosure, the promoter disclosed herein may be modified. Those skilled in the art can create promoters that have variations in the polynucleotide sequence. The polynucleotide sequence of the promoter of the present disclosure as shown in SEQ ID NO:1, 2, 3 or 4 may be modified or altered to enhance their control characteristics. As one of ordinary skill in the art will appreciate, modification or alteration of the promoter sequence can also be made without substantially affecting the promoter function. The methods are well known to those of skill in the art. Sequences can be modified, for example by insertion, deletion, or replacement of template sequences in a PCR-based DNA modification approach.

The present disclosure encompasses functional fragments and variants of the promoter sequence disclosed herein.

A “functional fragment” as used herein is any subset of contiguous nucleotides of the promoter sequence disclosed herein, that can perform the same, or substantially similar function as the full length promoter sequence disclosed herein. A “functional fragment” with substantially similar function to the full length promoter disclosed herein refers to a functional fragment that retains largely the same level of activity as the full length promoter sequence and exhibits the same pattern of expression as the full length promoter sequence.

A “variant”, as used herein, is the sequence of the promoter or the sequence of a functional fragment of a promoter containing changes in which one or more nucleotides of the original sequence is deleted, added, and/or substituted, while substantially maintaining promoter function. One or more base pairs can be inserted, deleted, or substituted internally to a promoter. In the case of a promoter fragment, variant promoters can include changes affecting the transcription of a minimal promoter to which it is operably linked. Variant promoters can be produced, for example, by standard DNA mutagenesis techniques or by chemically synthesizing the variant promoter or a portion thereof.

Substitutions, deletions, insertions or any combination thereof can be combined to produce a final construct.

“Enhancer sequences” refer to the sequences that can increase gene expression. These sequences can be located upstream, within introns or downstream of the transcribed region. The transcribed region is comprised of the exons and the intervening introns, from the promoter to the transcription termination region. The enhancement of gene expression can be through various mechanisms which include, but are not limited to, increasing transcriptional efficiency, stabilization of mature mRNA and translational enhancement.

An “intron” is an intervening sequence in a gene that is transcribed into RNA and then excised in the process of generating the mature mRNA. The term is also used for the excised RNA sequences. An “exon” is a portion of the sequence of a gene that is transcribed and is found in the mature messenger RNA derived from the gene, and is not necessarily a part of the sequence that encodes the final gene product.

Many genes exhibit enhanced expression on inclusion of an intron in the transcribed region, especially when the intron is present within the first 1 kb of the transcription start site. The increase in gene expression by presence of an intron can be at both the mRNA (transcript abundance) and protein levels. The mechanism of this Intron Mediated Enhancement (IME) in plants is not very well known (Rose et al., Plant Cell, 20: 543-551(2008) Le-Hir et al, Trends Biochem Sci. 28: 215-220 (2003), Buchman and Berg, Mol. Cell Biol. (1988) 8:4395-4405; Callis et al., Genes Dev. 1(1987):1183-1200).

An “enhancing intron” is an intronic sequence present within the transcribed region of a gene which is capable of enhancing expression of the gene when compared to an intronless version of an otherwise identical gene. An enhancing intronic sequence might also be able to act as an enhancer when located outside the transcribed region of a gene, and can act as a regulator of gene expression independent of position or orientation (Chan et. al. (1999) Proc. Natl. Acad. Sci. 96: 4627-4632; Flodby et al. (2007) Biochem. Biophys. Res. Commun. 356: 26-31).

An intron sequence can be added to the 5′ untranslated region, the protein-coding region or the 3′ untranslated region to increase the amount of the mature message that accumulates in the cytosol.

The intron sequences can be operably linked to a promoter and a gene of interest.

The present disclosure includes a recombinant construct comprising promoter functional in a plant cell, wherein the promoter comprises a nucleic acid sequence of at least 90%, 91%, 92%, 93%, 94%, 95%, 96%, 97%, 98%, 99%, or 100% sequence identity, based on the Clustal V method of alignment with default parameters of KTUPLE=2, GAP PENALTY=5, WINDOW=4 and DIAGONALS SAVED=4, when compared to SEQ ID NO:1, 2, 3 or 4. The promoter may comprise a nucleic acid sequence with less than 100% identity to SEQ ID NO:1, 2, 3 or 4.

The present disclosure includes a recombinant DNA construct comprising a promoter functional in a plant cell, wherein the promoter comprises the nucleic acid sequence given in SEQ ID NO:1, 2, 3 or 4.

The present disclosure also includes a recombinant DNA construct comprising a functional fragment of the promoter described herein, wherein the functional fragment of the promoter is functional in a plant cell, and drives the expression of an operably linked heterologous polynucleotide in the plant cell. Functional fragments of a promoter may be at least 100, 200, 300, 400, 500, 600, 700, 800, 900, 1000, 1500, 2000 nucleotides, and up to the full-length of the promoter.

The present disclosure includes a recombinant DNA construct comprising a promoter functional in a plant cell, wherein the promoter is a functional fragment of the promoter sequence given in SEQ ID NO:1, 2, 3 or 4, wherein the functional fragment of the promoter is functional in a plant cell, and drives the expression of an operably linked heterologous polynucleotide in the plant cell.

The present disclosure includes a recombinant DNA construct comprising a promoter functional in a plant cell, wherein the promoter is selected from the group consisting of: (a) a nucleotide sequence that is hybridizable under stringent conditions with a DNA molecule comprising the full complement of SEQ ID NO:1, 2, 3 or 4; (b) a nucleotide sequence that is derived from SEQ ID NO:1, 2, 3 or 4 by alteration of one or more nucleotides by at least one method selected from the group consisting of: deletion, substitution, addition and insertion; and (c) an allele of SEQ ID NO:1, 2, 3 or 4.

The present disclosure includes a recombinant DNA construct comprising promoter functional in a plant cell, wherein the promoter is a functional fragment of the promoter sequence that has at least 90%, 91%, 92%, 93%, 94%, 95%, 96%, 97%, 98%, 99%, or 100% sequence identity, based on the Clustal V method of alignment with default parameters of KTUPLE=2, GAP PENALTY=5, WINDOW=4 and DIAGONALS SAVED=4, when compared to SEQ ID NO:1, 2, 3 or 4, wherein the functional fragment of the promoter is functional in a plant cell, and drives the expression of an operably linked heterologous polynucleotide in the plant cell.

Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153, U.S. Pat. No. 8,399,736) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

The present disclosure encompasses an isolated polynucleotide that functions as a promoter in a plant, wherein the polynucleotide has a nucleotide sequence that can hybridize under stringent conditions with the nucleotide sequence of SEQ ID NO:1, 2, 3 or 4. The polynucleotide also may function as a constitutive promoter in a plant. The polynucleotide also may comprise at least 50, 100, 200, 300, 400, 500, 1000, 1500 or 2000 contiguous nucleotides of SEQ ID NO:1, 2, 3 or 4. The polynucleotide also may have at least 90%, 91%, 92%, 93%, 94%, 95%, 96%, 97%, 98%, 99%, or 100% sequence identity, based on the Clustal V method of alignment with default parameters of KTUPLE=2, GAP PENALTY=5, WINDOW=4 and DIAGONALS SAVED=4, when compared to SEQ ID NO:1, 2, 3 or 4.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Moderately stringent conditions may include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC; e.g., 6×SSC, or 2×SSC, or 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

The present disclosure also encompasses an isolated polynucleotide that functions as a promoter in a plant and comprises a nucleotide sequence that is derived from SEQ ID NO:1, 2, 3 or 4 by alteration of one or more nucleotides by at least one method selected from the group consisting of: deletion, substitution, addition and insertion. The polynucleotide also may function as a constitutive promoter in a plant. The polynucleotide also may comprise at least 50, 100, 200, 300, 400, 500, 1000, 1500 or 2000 contiguous nucleotides of SEQ ID NO:1, 2, 3 or 4. The polynucleotide also may have at least 90%, 91%, 92%, 93%, 94%, 95%, 96%, 97%, 98%, 99%, or 100% sequence identity, based on the Clustal V method of alignment with default parameters of KTUPLE=2, GAP PENALTY=5, WINDOW=4 and DIAGONALS SAVED=4, when compared to SEQ ID NO:1, 2, 3 or 4.

The present disclosure encompasses an isolated polynucleotide comprising a nucleotide sequence, wherein the nucleotide sequence corresponds to an allele of SEQ ID NO:1, 2, 3 or 4.

The present disclosure provides a recombinant DNA construct comprising a promoter functional in a plant cell operably linked to a heterologous polynucleotide, wherein the promoter comprises a nucleotide sequence selected from the group consisting of: (a) a nucleotide sequence with at least 90%, 91%, 92%, 93%, 94%, 95%, 96%, 97%, 98%, 99% or 100% sequence identity to SEQ ID NO:1, 2, 3 or 4, using a Clustal V method of alignment with pairwise alignment default parameters of KTUPLE=2, GAP PENALTY=5, WINDOW=4 and DIAGONALS SAVED=4; (b) a nucleotide sequence comprising a functional fragment of SEQ ID NO:1, 2, 3 or 4, wherein the functional fragment comprises at least 100, 200, 300, 400, 500, 1000, 1500, or 2000 contiguous nucleotides of SEQ ID NO:1, 2, 3 or 4; and (c) SEQ ID NO:1, 2, 3 or 4; wherein the promoter drives expression of the heterologous polynucleotide in a plant cell.

The promoter of the recombinant DNA construct may drive expression in at least one tissue selected from the group consisting of: anther, leaf, root and stalk. The promoter of the recombinant DNA construct may drive expression in anther, leaf, root and stalk.

The promoter of the recombinant DNA construct may drive expression of the heterologous polynucleotide primarily in plant vascular cells.

The promoter and the heterologous polynucleotide of the recombinant DNA construct may each be operably linked to an intron.

The promoter of the recombinant DNA construct may be operably linked to an enhancer, or may be operably linked to both an intron and an enhancer.

Alternatively, the promoter of the recombinant DNA construct may not be operably linked to a heterologous enhancer.

The present disclosure also includes a plant or seed comprising the recombinant DNA construct. The plant or seed may be a monocot plant or monocot seed. The monocot plant or monocot seed may be a maize plant or maize seed. The present disclosure provides a method of making a transgenic plant, the method comprising the steps of: (a) introducing into a regenerable plant cell the recombinant DNA construct of the present disclosure; and (b) regenerating a transgenic plant from the regenerable plant cell after step (a), wherein the transgenic plant comprises the recombinant DNA construct. The method may further comprise growing the transgenic plant of step (b) under conditions in which the heterologous polynucleotide is expressed. The method may further comprise growing a progeny plant derived from the transgenic plant of step (b), wherein said progeny plant comprises the recombinant DNA construct and wherein the progeny plant is grown under conditions in which the heterologous polynucleotide is expressed. In any of the methods of the present disclosure, the plant may be a monocot plant. Additionally, the monocot plant may be a maize plant.

The present disclosure also provides a plant comprising in its genome an endogenous polynucleotide operably linked to at least one heterologous promoter functional in a plant cell, wherein the promoter comprises a nucleotide sequence selected from the group consisting of: (a) a nucleotide sequence with at least 90%, 91%, 92%, 93%, 94%, 95%, 96%, 97%, 98%, 99% or 100% sequence identity to SEQ ID NO:1, 2, 3 or 4, using a Clustal V method of alignment with pairwise alignment default parameters of KTUPLE=2, GAP PENALTY=5, WINDOW=4 and DIAGONALS SAVED=4; (b) a nucleotide sequence comprising a functional fragment of SEQ ID NO:1, 2, 3 or 4, wherein the functional fragment comprises at least 100, 200, 300, 400, 500, 1000, 1500, or 2000 contiguous nucleotides of SEQ ID NO:1, 2, 3 or 4; and (c) SEQ ID NO:1, 2, 3 or 4; wherein said plant exhibits at least one trait selected from the group consisting of: increased drought tolerance, increased yield, increased biomass, and altered root architecture, when compared to a control plant not comprising the heterologous regulatory element.

The present disclosure also provides for the use of any of the recombinant DNA constructs of the present disclosure to produce a plant that exhibits at least one trait selected from the group consisting of: increased drought tolerance, increased yield, increased biomass, and altered root architecture, when compared to a control plant not comprising the heterologous regulatory element.

A method of selecting for an alteration of an agronomic characteristic in a plant, comprising (a) obtaining a transgenic plant, wherein the transgenic plant comprises in its genome a recombinant DNA construct comprising a promoter as described in the present disclosure operably linked to at least one heterologous polynucleotide (for example, a polynucleotide encoding a protein that conveys stress tolerance in a plant); and (b) selecting a transgenic plant of (a), wherein the polynucleotide is expressed in the plant, and further wherein the plant exhibits an alteration of at least one agronomic characteristic when compared, optionally under water limiting conditions, to a control plant not comprising the recombinant DNA construct.

A method of producing a plant of the present disclosure, wherein the method comprises growing a plant from a seed comprising a recombinant DNA construct, wherein the recombinant DNA construct comprises a promoter as described in the present disclosure operably linked to at least one heterologous polynucleotide, wherein the plant exhibits at least one trait selected from the group consisting of: increased drought tolerance, increased yield, increased biomass, and altered root architecture, when compared to a control plant not comprising the recombinant DNA construct. The plant may be selected from the group consisting of: maize, soybean, sunflower, sorghum, canola, wheat, alfalfa, cotton, rice, barley, millet, sugar cane and switchgrass.

A method of producing a seed, the method comprising: (a) crossing a first plant with a second plant, wherein at least one of the first plant and the second plant comprises a recombinant DNA construct, wherein the recombinant DNA construct comprises a promoter as described in the present disclosure operably linked to at least one heterologous polynucleotide; and (b) selecting a seed of the crossing of step (a), wherein the seed comprises the recombinant DNA construct. A plant grown from the seed may exhibit at least one trait selected from the group consisting of: increased drought tolerance, increased yield, increased biomass and altered root architecture, when compared to a control plant not comprising the recombinant DNA construct. The plant may be selected from the group consisting of: maize, soybean, sunflower, sorghum, canola, wheat, alfalfa, cotton, rice, barley, millet, sugar cane and switchgrass.

A method of producing seed (for example, seed that can be sold as a drought tolerant product offering) comprising any of the preceding methods, and further comprising obtaining seeds from said progeny plant, wherein said seeds comprise in their genome said recombinant DNA construct.

A method of producing oil or a seed by-product, or both, from a seed, the method comprising extracting oil or a seed by-product, or both, from a seed that comprises a recombinant DNA construct, wherein the recombinant DNA construct comprises a promoter as described in the present disclosure operably linked to at least one heterologous polynucleotide. The seed may be obtained from a plant that comprises the recombinant DNA construct, wherein the plant exhibits at least one trait selected from the group consisting of: increased drought tolerance, increased yield, increased biomass, and altered root architecture, when compared to a control plant not comprising the recombinant DNA construct. The plant may be selected from the group consisting of: maize, soybean, sunflower, sorghum, canola, wheat, alfalfa, cotton, rice, barley, millet, sugar cane and switchgrass. The oil or the seed by-product, or both, may comprise the recombinant DNA construct.

Methods of isolating seed oils are well known in the art: (Young et al., Processing of Fats and Oils, In The Lipid Handbook, Gunstone et al., eds., Chapter 5 pp 253 257; Chapman & Hall: London (1994)). Seed by-products include but are not limited to the following: meal, lecithin, gums, free fatty acids, pigments, soap, stearine, tocopherols, sterols and volatiles.

One may evaluate altered root architecture in a controlled environment (e.g., greenhouse) or in field testing. The evaluation may be under simulated or naturally-occurring water limiting conditions. The altered root architecture may be an increase in root mass. The increase in root mass may be at least 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45% or 50%, when compared to a control plant not comprising the recombinant DNA construct.

A method is provided of making a plant, wherein the method comprises: (a) introducing into a plant cell a double-stranded break-inducing agent and at least one heterologous₌promoter functional in a plant cell, wherein the promoter comprises a nucleotide sequence selected from the group consisting of: (i) a nucleotide sequence with at least 90%, 91%, 92%, 93%, 94%, 95%, 96%, 97%, 98%, 99% or 100% sequence identity to SEQ ID NO:1, 2, 3 or 4, using a Clustal V method of alignment with pairwise alignment default parameters of KTUPLE=2, GAP PENALTY=5, WINDOW=4 and DIAGONALS SAVED=4; (ii) a nucleotide sequence comprising a functional fragment of SEQ ID NO:1, 2, 3 or 4, wherein the functional fragment comprises at least 100, 200, 300, 400, 500, 1000, 1500, or 2000 contiguous nucleotides of SEQ ID NO:1, 2, 3 or 4; and (iii) SEQ ID NO:1, 2, 3 or 4; (b) regenerating a plant from the plant cell of step (a); (c) selecting a plant from step (b) that comprises the heterologous promoter operably linked an endogenous protein-coding sequence. Optionally, the method also consisting of: (d) selecting the plant of step (c) that exhibits at least one trait selected from the group consisting of: increased drought tolerance, increased yield, increased biomass, and altered root architecture, when compared to a control plant not comprising the heterologous promoter operably linked to the endogenous protein-coding sequence. The plant may be selected from the group consisting of: maize, soybean, sunflower, sorghum, canola, wheat, alfalfa, cotton, rice, barley, millet, sugar cane and switchgrass.

In any of the preceding methods or any other methods of the present disclosure, the at least one agronomic characteristic (or trait) may be selected from the group consisting of: abiotic stress tolerance, greenness, stay-green, yield, growth rate, biomass, fresh weight at maturation, dry weight at maturation, fruit yield, seed yield, seed size, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen stress tolerance, nitrogen uptake, standability, root lodging, root architecture, root mass, average root length, leaf size, harvest index, stalk lodging, plant height, ear height, ear length, ear size, endosperm size, embryo size, salt tolerance, early seedling vigor and seedling emergence under low temperature stress. The alteration of at least one agronomic characteristic may be an increase, e.g., in drought tolerance, yield, stay-green or biomass (or any combination thereof), or a decrease, e.g., in root lodging.

In any of the preceding methods or any other methods of the present disclosure, the step of selecting for an alteration of an agronomic characteristic in a transgenic plant (or progeny plant), if applicable, may comprise selecting a transgenic plant (or progeny plant) that exhibits an alteration of at least one agronomic characteristic when compared, under varying environmental conditions, e.g., under water limiting conditions, to a control plant not comprising the recombinant DNA construct.

In any of the preceding methods or any other methods of the present disclosure, in said introducing step said regenerable plant cell may comprise a callus cell, an embryogenic callus cell, a gametic cell, a meristematic cell, or a cell of an immature embryo. The regenerable plant cells may derive from an inbred maize plant.

In any of the preceding methods or any other methods of the present disclosure, said regenerating step may comprise: (i) culturing said transformed plant cells in a media comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

The compositions and methods of the present disclosure can be used in dicots or monocots. In particular, the compositions and methods of the present disclosure can be used in maize.

The present disclosure includes transformed plant cells, tissues, plants, and seeds. Also included are the following: regenerated, mature and fertile transgenic plants; transgenic seeds produced therefrom; and T1 and subsequent generations. The transgenic plant cells, tissues, plants, and seeds may comprise at least one recombinant DNA construct of interest.

The present disclosure includes a transgenic microorganism or cell comprising the recombinant DNA construct. The microorganism or cell may be eukaryotic, e.g., a yeast, insect or plant cell, or prokaryotic, e.g., a bacterial cell.

Sequence changes can be introduced at specific selected sites using double-strand-break technologies such as ZNFs, custom designed homing endonucleases, TALENs, CRISPR/CAS (also referred to as guide RNA/Cas endonuclease systems; See U.S. Patent Application Publication No. 2015/0082478, herein incorporated by reference), or other protein and/or nucleic acid based mutagenesis technologies. The resultant variants can be screened for altered activity. It will be appreciated that these techniques are often not mutually exclusive. Indeed, the various methods can be used singly or in combination, in parallel or in series, to create or access diverse sequence variants.

CRISPR loci (Clustered Regularly Interspaced Short Palindromic Repeats; also known as SPIDRs-SPacer Interspersed Direct Repeats) constitute a family of DNA loci. CRISPR loci consist of short and highly conserved DNA repeats (typically 24 to 40 base pairs, repeated from 1 to 140 times; also referred to as CRISPR-repeats) which are partially palindromic. The repeated sequences (usually specific to a species) are interspaced by variable sequences of constant length (typically 20 to 58 base pairs depending on the CRISPR locus; See WO2007/025097 published Mar. 1, 2007, herein incorporated by reference).

Cas endonuclease relates to a Cas protein encoded by a Cas gene, wherein said Cas protein is capable of introducing a double strand break into a DNA target sequence. The Cas endonuclease is guided by a guide polynucleotide to recognize and optionally introduce a double strand break at a specific target site into the genome of a cell (See U.S. Patent Application Publication No. 2015/0082478; herein incorporated by reference). The guide polynucleotide/Cas endonuclease system includes a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide RNA if a correct protospacer-adjacent motif (PAM) is approximately oriented at the 3′ end of the target sequence.

The introduction of recombinant DNA constructs of the present disclosure into plants may be carried out by any suitable technique, including but not limited to direct DNA uptake, chemical treatment, electroporation, microinjection, cell fusion, infection, vector mediated DNA transfer, bombardment, or Agrobacterium mediated transformation. Techniques for plant transformation and regeneration have been described in International Patent Publication WO 2009/006276, the contents of which are herein incorporated by reference.

The development or regeneration of plants containing the foreign, exogenous polynucleotide of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

### EXAMPLES

The present disclosure is further illustrated in the following Examples, in which parts and percentages are by weight and degrees are Celsius, unless otherwise stated. It should be understood that these examples are given by way of illustration only. From the above discussion and these Examples, one skilled in the art can ascertain the essential characteristics of the present disclosure, and without departing from the spirit and scope thereof, can make various changes and modifications of the present disclosure to adapt it to various usages and conditions. Furthermore, various modifications of this disclosure in addition to those shown and described herein will be apparent to those skilled in the art from the foregoing description. Such modifications are also intended to fall within the scope of the appended claims.

### Example 1

### Cloning of Zea mays and Sorghum bicolor S2A Promoter Sequences

The Medicago sativa S-Adenosyl-Homocysteine Hydrolase (MS-S2A) promoter that shows vascular-specific expression pattern has been previously tested in maize using a selectable marker that contained a 35S enhancer (Abrahams et al Plant Molecular Biology 27: 513-528, 1995). When this cassette was retested without the 35S enhancer, the promoter did not function (Table 1).

To determine if related promoters could function without being operably linked to a 35S enhancer, orthologous promoters from monocots (Sorghum and maize) were tested.

For testing the orthologous promoters from corn and sorghum, the Zea mays S-Adenosyl-Homocysteine Hydrolase (ZM-S2A) and Sorghum bicolor S-Adenosyl-Homocysteine Hydrolase (SB-S2A) promoter sequences were synthesized (GENSCRIPT®), using available genomic sequence information.

For the Sorghum bicolor S2A promoter, approximately 2.5 kb of the sequence upstream of the translation start site was synthesized (SEQ ID NO:1), and cloned into a GUS entry clone PHP56634. The entry clone PHP56634 contains an attL4 site (SEQ ID NO:5), into which the promoters to be tested were inserted. The attL4 site in PHP56634 is followed by a “GUSINT” region (SEQ ID NO:6); i.e., a β-glucuronidase coding region (GUS) that has been interrupted with an intron in order to prevent GUS expression in bacteria. The GUSINT region is followed by a potato pinII terminator (SEQ ID NO:7) and a Sorghum actin terminator (SEQ ID NO:8).

PHP64037, containing the SB-S2A promoter (SEQ ID NO:1) upstream of the GUS gene, was used for further transformation into corn plants. The Sorghum bicolor S2A promoter sequence cloned into PHP64037 included 81 bp of 5′UTR. SEQ ID NO:1 is the sequence of the SB-S2A promoter including the 5′UTR, and SEQ ID NO:3 is the sequence of the SB-S2A promoter upstream of the predicted transcription start site (TSS).

For Zea mays S2A promoter, approximately 2.5 kb of the sequence upstream of the translation start site was synthesized (SEQ ID NO:2), and cloned into a GUS entry clone PHP56634 to give the plasmid PHP64062. PHP64062, containing the ZM-S2A promoter (SEQ ID NO:2) upstream of the GUS gene, was used for further transformation into corn plants. The Zea mays cloned S2A promoter sequence included 83 bp of 5′UTR. SEQ ID NO:2 is the sequence of the ZM-S2A promoter including the 5′UTR, and SEQ ID NO:4 is the sequence of the ZM-S2A promoter upstream of the predicted transcription start site (TSS).

No heterologous enhancer sequence was included in the plasmids constructed to test the activity of the ZM-S2A and the SB-S2A promoters.

### Example 2

### ZM-S2A and SB-S2A Promoter Activity

The Agrobacterium transformation vectors PHP64037 with the SB-S2A promoter (SEQ ID NO:1) and PHP64062 with the ZM-S2A promoter (SEQ ID NO:2) described in Example 1, were used in Gaspe-Flint derived maize lines for stable transformation to generate transgenic maize plants. The MS-S2A promoter, in the absence of an enhancer, was tested with the construct PHP51842. Maize transformation was done by standard procedures (US Patent Publication No. US2014/0068811, herein incorporated by reference).

Both qualitative and quantitative GUS reporter gene expression analyses were carried out on at least 4 independent single copy events. Different tissue samples were collected for histochemical GUS staining with 5-bromo-4-chloro-3-indolyl-β-D-glucuronide (X-Gluc), using standard protocols (Janssen and Gardner, Plant Mol. Biol. (1989) 14:61-72) and for quantitative MUG assay using standard protocols (Jefferson, R. A., Nature. 342, 837-8 (1989); Jefferson, R. A., Kavanagh, T. A. & Bevan, M. W., EMBO J. 6, 3901-3907 (1987).

GUS reporter gene expression was determined in T0 corn plants. The tissues tested for GUS reporter gene expression were leaf (V6 stage), root (VT stage), anther (VT stage) and Stalk (R1 stage).

As can be seen in Table 1, the MS-S2A promoter didn't drive GUS gene expression in any of the tested tissues, in the absence of an enhancer.

TABLE 1
GUS MUG Scores for Events Containing the MS-S2A Promoter

| PHP51842 EVENT | ANTHER | LEAF | ROOT | STALK |
|---|---|---|---|---|
| EA_1_MS-S2A PRO | 0 | 0 | 0 | 0 |
| EA_2_MS-S2A PRO | 0 | 0 | 0 | 0 |
| EA_3_MS-S2A PRO | 0 | 0 | 0 | 0 |
| EA_4_MS-S2A PRO | 0 | 0 | 0 | 0 |
| EA_5_MS-S2A PRO | 0 | 0 | 0 | 0 |
| EA_6_MS-S2A PRO | 0 | 0 | 0 | 0 |
| EA_7_MS-S2A PRO | 0 | 0 | 0 | 0 |
| EA_8_MS-S2A PRO | 0 | 0 | 0 | 0 |
| EA_9_MS-S2A PRO | 0 | 0 | 0 | 0 |
| EA_10_MS-S2A PRO | 0 | 0 | 0 | 0 |

For the SB-S2A promoter, GUS reporter gene expression was observed in the vascular tissue in stalk, roots, and anthers from T0 corn events, and some GUS expression was also observed in leaves (assays were done with plants cultivated in the greenhouse). The MUG values are given in Table 2

TABLE 2
GUS MUG Scores for Events Containing the SB-S2A Promoter

| PHP64037 EVENT | ANTHER | LEAF | ROOT | STALK |
|---|---|---|---|---|
| EA_1_SB-S2A_PRO | 674 | 25.9 | 30.5 | 87.7 |
| EA_2_SB-S2A_PRO | 877 | 29.7 | 158 | 166 |
| EA_3_SB-S2A_PRO | 713 | 27.9 | 39.9 | 115 |
| EA_4_SB-S2A_PRO | 749 | 23.9 | 47.9 | 156 |
| EA_5_SB-S2A_PRO | 817 | 45.5 | 60.3 | 222 |
| EA_6_SB-S2A_PRO | 739 | 34.3 | 158 | 217 |
| EA_7_SB-S2A_PRO | 758 | 42.4 | 76.3 | 175 |
| EA_8_SB-S2A_PRO | 871 | 48.3 | 90.1 | 199 |

For ZM-S2A promoter, GUS reporter gene expression was observed in the vascular tissue in stalk, roots, and anthers from T0 corn events, and some GUS expression was also observed in leaves (assays were done with plants cultivated in the greenhouse). The MUG values are given in Table 3.

TABLE 3
GUS MUG Scores for Events Containing the ZM-S2A Promoter

| PHP64062 EVENT | ANTHER | LEAF | ROOT | STALK |
|---|---|---|---|---|
| EA_1_ZM_S2A_PRO | 241 | — | 88.8 | 66.1 |
| EA_2_ZM_S2A_PRO | 652 | 22.4 | 91.4 | 0 |
| EA_3_ZM_S2A_PRO | 266 | 79 | 491 | 287 |
| EA_4_ZM_S2A_PRO | 563 | 47.7 | 106 | 134 |
| EA_5_ZM_S2A_PRO | 202 | 24.6 | 312 | 201 |
| EA_6_ZM_S2A_PRO | 255 | 32.3 | 294 | 188 |
| EA_7_ZM_S2A_PRO | 114 | 28.9 | 231 | 105 |

Transformed plants containing the SB-S2A::GUS expression cassette or the ZM-S2A::GUS expression cassette were also compared to corresponding transformed plants containing the ZM-Ubi1::GUS expression cassette or the ZM-GOS2::GUS expression cassette. In each expression vector the GUS coding region (SEQ ID NO:6) is followed by a potato pinII terminator (SEQ ID NO:7) and a Sorghum actin terminator (SEQ ID NO:8). The ZM-Ubi1 promoter and intron used is presented in SEQ ID NO:9. The ZM-GOS2 promoter and intron used is presented in SEQ ID NO:10. Leaf tissue from maize plants transformed with each of the four GUS expression cassettes was stained and examined for GUS expression. GUS expression vectors having the ZM-UBI1 promoter and intron, or the ZM-GOS2 promoter and intron, were observed to have constitutive GUS expression in maize leaf tissues; however, GUS expression vectors having the SB-S2A promoter or the ZM-S2A promoter were observed to have vascular-preferred GUS gene expression in the leaf.

### Example 3

### ZM-S2A and SB-S2A Promoter Activity in the Presence of Other Regulatory Elements

The ZM-S2A and the SB-S2A promoters may be cloned with other regulatory elements, such as introns and/or enhancers, to modulate gene expression driven by these promoters.

### Example 4

### ZM-S2A Promoter Operably Linked to Heterologous Introns

The ZM-S2A promoter was used, either with or without various heterologous maize introns, to drive expression of polynucleotides encoding the following proteins having stress tolerant (ST) activity in maize: ST-A, ST-B, ST-C and ST-D. The following introns were used in the various maize expression vectors: ZM-ADH1 Intron 1 (SEQ ID NO: 11), ZM-HPLV9 Intron1 (SEQ ID NO:12) and ZM-TA4 Intron1 (SEQ ID NO:13). ZM-HPLV9 Intron1 and ZM-TA4 Intron1 correspond to SEQ ID NO:8 and SEQ ID NO:138, respectively, in PCT International Patent Publication No. WO02011156535, herein incorporated by reference. The promoter, intron and protein-coding regions (CDS) of each vector are presented in Table 4.

TABLE 4
Expression Vectors for Stress Tolerant Genes

| Vector | Promoter | Intron | CDS |
|---|---|---|---|
| pZMS2A-1 | ZM-S2A | ZM-HPLV9 INTRON1 | ST-A |
| pZMS2A-2 | ZM-S2A | ZM-ADH1 INTRON1 | ST-B |
| pZMS2A-3 | ZM-S2A | no intron | ST-C |
| pZMS2A-4 | ZM-S2A | ZM-ADH1 INTRON1 | ST-C |
| pZMS2A-5 | ZM-S2A | ZM-TA4 INTRON1 | ST-D |
| pZMS2A-6 | ZM-S2A | no intron | ST-B |
| pZMS2A-7 | ZM-S2A | no intron | ST-D |
| pZMS2A-8 | ZM-S2A | ZM-HPLV9 INTRON1 | ST-B |
| pZMS2A-9 | ZM-S2A | no intron | ST-C |
| pZMS2A-10 | ZM-S2A | ZM-ADH1 INTRON1 | ST-D |
| pZMS2A-11 | ZM-S2A | ZM-TA4 INTRON1 | ST-A |

Maize plants were transformed with each of the vectors listed in Table 4 and the resulting transgenic plants were grown under field conditions.

We claim:

1. A recombinant DNA construct comprising a promoter functional in a plant cell operably linked to a heterologous polynucleotide, wherein the promoter comprises a nucleotide sequence selected from the group consisting of:
(a) a nucleotide sequence with at least 90% sequence identity to SEQ ID NO:1, 2, 3 or 4, using a Clustal V method of alignment with pairwise alignment default parameters of KTUPLE=2, GAP PENALTY=5, WINDOW=4 and DIAGONALS SAVED=4;
(b) a nucleotide sequence comprising a functional fragment of SEQ ID NO:1, 2, 3 or 4, wherein the functional fragment comprises at least 100 contiguous nucleotides of SEQ ID NO:1, 2, 3 or 4; and
(c) SEQ ID NO:1, 2, 3 or 4;
wherein the promoter drives expression of the heterologous polynucleotide in a plant cell.

2. The recombinant DNA construct of claim 1, wherein the promoter drives expression in at least one tissue selected from the group consisting of: anther, leaf, root and stalk.

3. The recombinant DNA construct of claim 2, wherein the promoter drives expression in anther, leaf, root and stalk.

4. The recombinant DNA construct of claim 1, wherein the promoter drives the expression of the heterologous polynucleotide primarily in plant vascular cells.

5. The recombinant DNA construct of claim 1, wherein the promoter and the heterologous polynucleotide are each operably linked to an intron.

6. The recombinant DNA construct of claim 1, wherein the promoter is not operably linked to a heterologous enhancer.

7. A plant or seed comprising the recombinant DNA construct of claim 1.

8. The plant or seed of claim 7, wherein the plant or seed is a monocot plant or monocot seed.

9. The monocot plant or monocot seed of claim 8, wherein the monocot plant or monocot seed is a maize plant or maize seed.

10. A method of making a transgenic plant, the method comprising the steps of:
(a) introducing into a regenerable plant cell the recombinant DNA construct of claim 1; and
(b) regenerating a transgenic plant from the regenerable plant cell after step (a), wherein the transgenic plant comprises the recombinant DNA construct.

11. The method of claim 10, further comprising growing the transgenic plant of step (b) under conditions in which the heterologous polynucleotide is expressed.

12. The method of claim 10, further comprising growing a progeny plant derived from the transgenic plant of step (b), wherein said progeny plant comprises the recombinant DNA construct and wherein the progeny plant is grown under conditions in which the heterologous polynucleotide is expressed.

13. The method of claim 10, wherein the plant is a monocot plant.

14. The method of claim 13, wherein the monocot plant is a maize plant.

15. A plant comprising in its genome an endogenous polynucleotide operably linked to at least one heterologous promoter functional in a plant cell, wherein the promoter comprises a nucleotide sequence selected from the group consisting of:
(a) a nucleotide sequence with at least 90% sequence identity to SEQ ID NO:1, 2, 3 or 4, using a Clustal V method of alignment with pairwise alignment default parameters of KTUPLE=2, GAP PENALTY=5, WINDOW=4 and DIAGONALS SAVED=4;
(b) a nucleotide sequence comprising a functional fragment of SEQ ID NO:1, 2, 3 or 4, wherein the functional fragment comprises at least 100 contiguous nucleotides of SEQ ID NO:1, 2, 3 or 4; and
(c) SEQ ID NO:1, 2, 3 or 4;
wherein said plant exhibits at least one trait selected from the group consisting of: increased drought tolerance, increased yield, increased biomass, and altered root architecture, when compared to a control plant not comprising the heterologous regulatory element.

16. Use of the recombinant DNA construct of claim 1 to produce a plant that exhibits at least one trait selected from the group consisting of: increased drought tolerance, increased yield, increased biomass, and altered root architecture, when compared to a control plant not comprising the heterologous regulatory element.
